(19) United States
(12) Patent Application Publication
(10) Pub. No.: US 2003/0082221 A1
(43) Pub. Date: May 1, 2003

(54) Pullulan based film forming cosmetic compositions

(75) Inventors: David P. O'Halloran, Milltown, NJ (US); Yelena Zolotarsky, Springfield, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Lavipharm Laboratories Inc.

(21) Appl. No.: 10/256,234

(22) Filed: Sep. 26, 2002

Related U.S. Application Data
(60) Provisional application No. 60/325,221, filed on Sep. 27, 2001.

(51) Int. Cl. A61K 7/021; A61K 7/00

(52) U.S. Cl. 424/401; 424/63

(57) ABSTRACT

Compositions for forming a visible and distinctive cosmetic film on the skin of a subject are provided. The compositions provided contain 3 to 15 wt % pullulan. The invention also relates to a method of forming a visible and distinctive cosmetic film on the skin of a subject including applying a composition to the skin of the subject, wherein the composition contains 3 to 15 wt % pullulan.

This application claims priority from U.S. Provisional Application Serial No. 60/325,221 filed Sep. 27, 2001; the disclosure of which is incorporated herein by reference as if set forth in its entirety.

The present invention relates to compositions for forming visible and distinctive cosmetic films on the skin of a subject. More particularly, the present invention relates to compositions for forming visible and distinctive cosmetic films on the skin of a subject, containing 3 to 15 wt % pullulan. The present invention also relates to a method for forming a visible and distinctive cosmetic film on the skin of a subject including applying a composition of the present invention to the skin of the subject, wherein the composition contains 3 to 15 wt % pullulan.

Cosmetic films in are conventionally used to produce a variety of favorable results for the user. Two general categories of cosmetic films are: peel-off films and a rinse-away films. Both types of cosmetic films require that a coating be formed over the contours of the skin to which it is applied, typically on the face. The peel-off films are removed by physically peeling the dried film away from the skin and the rinse-away films are removed by thorough rinsing with water.

The major function of most conventional, cosmetic films is to deep clean facial skin by removing dead skin cells. Conventional, cosmetic films are sometimes believed to improve capillary blood circulation, cleanse, retexturize, firm and moisturize as well as stimulate the skin and increase cell regeneration. Moreover, conventional, cosmetic films are believed to tone the skin and their use is often believed to be beneficial exercise for the face.

### SUMMARY OF THE INVENTION

In a preferred embodiment of the present invention, compositions for forming a visible and distinctive cosmetic film on the skin of a subject are provided, containing 3 to 15 wt % pullulan, more preferably 3 to 10 wt % pullulan, most preferably about 10 wt % pullulan. In a preferred aspect of the invention, the composition for forming a visible and distinctive cosmetic film is provided as a semi-liquid gel or as a roll-on. Preferably, the compositions of the present invention are cosmetically elegant in nature, they spread easily onto the skin and are non-sticky.

In a preferred aspect of the present invention, the compositions for forming a visible and distinctive cosmetic film on the skin of a subject contain pullulan having a molecular weight of 200,000 to 2,000,000 Daltons.

In another preferred aspect of the present invention, the compositions for forming a visible and distinctive cosmetic film on the skin of a subject contain 20 to 97 wt % solvent. Preferably, the solvent is selected from the group of water, alcohol, glycol and mixtures thereof.

In another preferred aspect of the present invention, the compositions for forming a visible and distinctive cosmetic film on the skin of a subject contain 0.01 to 10 wt % active agent. Preferably, the active agent is selected from the group of alpha- and beta-hydroxy acids and derivatives thereof; vitamins and the salts, esters, alcohols and acid forms thereof; marine products and their derivatives; and topical over the counter active ingredients.

In another preferred aspect of the present invention, the compositions for forming a visible and distinctive cosmetic film on the skin of a subject contain 0.5 to 20 wt % humectant. Preferably, the humectant is selected from the group of glycols, sorbitol, sodium polycarboxcylic acid, sodium citrate and mixtures thereof.

In another preferred aspect of the present invention, the compositions for forming a visible and distinctive cosmetic film on the skin of the subject contain 0.1 to 10 wt % other polymers and thickener. Preferably, the polymers and thickener are selected from the group of xanthan gum, cellulose, polyvinyl pyrolidone, carbomer and mixtures thereof. The thickener may preferably be selected from the following group of mixtures, namely: a mixture of ammonium polyacrylate, isohexadecane and polyethylene glycol-40 castor oil; a mixture of polyacrylamide, polydecene, and ethoxylated lauryl alcohol; a mixture of polyacrylamide, C₁₃₋₁₄ isoparaffin, and ethoxylated lauryl alcohol; and, polyquarternium 32, and mineral oil.

In another preferred aspect of the present invention, the compositions for forming a visible and distinctive cosmetic film on the skin of the subject contain 0.01 to 10 wt % pigment. Preferably, the pigment is selected from the group of titanium dioxide, zinc oxide, iron oxide, Drug and Cosmetic (D&C) dyes, metalic lakes of cosmetic dyes and Food, Drug and Cosmetic (FD&C).

In another preferred aspect of the present invention, the compositions for forming a visible and distinctive cosmetic film on the skin of the subject contain 0.5 to 10 wt % emulsifier. Preferably, the emulsifier is selected from the group of ethoxylated alcohols, sodium lauryl sulfate, and polyquaternium-31.

In another preferred aspect of the present invention, the compositions for forming a visible and distinctive cosmetic film on the skin of the subject contain 0.5 to 10 wt % hydrocarbon. Preferably, the hydrocarbon is selected from the group of iso-paraffin, mineral oil, isododecane, almond oil and other natural oils.

In another preferred aspect of the present invention, the compositions for forming a visible and distinctive cosmetic film on the skin of the subject contain 0.1 to 5 wt % fragrance.

In another preferred aspect of the present invention, the compositions for forming a visible and distinctive cosmetic film on the skin of the subject contain 0.01 to 1 wt % chelating agent. Preferably, the chelating agent is selected from the group of ethylenediaminetetraacetic acid, ethylenediaminetetraacetic acid-disodium salt and ethylenediaminetetraacetic acid-tetrasodium salt.

In another preferred aspect of the present invention, the compositions for forming a visible and distinctive cosmetic film on the skin of the subject contain 0.1 to 4 wt % UV absorber. Preferably, the UV absorber is selected from the group of benzophenone-3 and benzophenone-4.

In another preferred aspect of the present invention, the compositions for forming a visible and distinctive cosmetic film on the skin of the subject contain 0.05 to 3 wt % antioxidant. Preferably, the antioxidant is selected from the group of tocopherol, tocopherol acetate, propyl gallate, butylated hydroxyanisole, and butylated hydroxytoluene.

In another preferred aspect of the present invention, the compositions for forming a visible and distinctive cosmetic film on the skin of the subject contain 0.2 to 3 wt % preservative. Preferably, the preservative is selected from the group of methylparaben, propylparaben and phenoxyethanol.

In another preferred embodiment of the present invention, compositions for forming a visible and distinctive cosmetic film on the skin of a subject are provided, containing: 3 to 15 wt % pullulan, 20 to 97 wt % solvent, 0.01 to 10 wt % active agent, 0.5-20 wt % humectant, 0.1 to 10 wt % other polymers and thickener, 0.01 to 10 wt % pigment, 0.5 to 10 wt % emulsifier, 0.5 to 10 wt % hydrocarbon, 0.1 to 5 wt % fragrance, 0.01 to 1 wt % chelating agent, 0.1 to 4 wt % UV absorber, 0.05 to 3 wt % antioxidant and 0.01 to 3 wt % preservative. In a preferred aspect of the invention, the composition for forming a visible and distinctive cosmetic film is provided as a semi-liquid gel or as a roll-on.

In another preferred embodiment of the present invention, a method for forming a visible and distinctive cosmetic film on the skin of a subject is provided, which method includes: applying to the skin of the subject a composition containing: 3 to 15 wt % pullulan, more preferably 3 to 10 wt % pullulan, most preferably about 10 wt % pullulan. In a preferred aspect of this embodiment of the present invention, the method for forming a visible and distinctive cosmetic film on the skin of a subject is provided, which method includes: applying to the skin of the subject a composition containing: 3 to 15 wt % pullulan, 20 to 97 wt % solvent, 0.01 to 10 wt % active agent, 0.5-20 wt % humectant, 0.1 to 10 wt % other polymers and thickener, 0.01 to 10 wt % pigment, 0.5 to 10 wt % emulsifier, 0.5 to 10 wt % hydrocarbon, 0.1 to 5 wt % fragrance, 0.01 to 1 wt % chelating agent, 0.1 to 4 wt % UV absorber, 0.05 to 3 wt % antioxidant and 0.01 to 3 wt % preservative.

### BRIEF DESCRIPTION OF THE DRAWING

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 is a graphical depiction of the skin permeation profile for a salycilic acid containing composition of the present invention.

### DETAILED DESCRIPTION

The present invention provides compositions for forming a comfortable, cosmetically elegant, visible and distinctive film on the skin of a subject. The unique pullulan containing compositions of the present invention dry to form cosmetically elegant, visible and distinctive films when applied to the skin of a subject. Particularly, the compositions of the present invention when applied to the skin of a subject form into a visible and distinctive film on the skin as the volatile components of the compositions evaporate. The resultant film is comfortable and can preferably be worn on the skin of the subject for a period of 20 minutes to several hours. After the composition dries into a film, it may be rinsed off the skin of the subject.

The compositions provided by the present invention for forming a visible and distinctive cosmetic film on the skin of a subject preferably contain 3 to 15 wt % pullulan, more preferably 3 to 10 wt % pullulan, most preferably about 10 wt % pullulan; and 20 to 97 wt % solvent. It has been found that formulations which contain less than around 3 wt % pullulan fail to produce an acceptable visible and distinctive cosmetic film. Conversely, formulations containing greater than about 15 wt % pullulan are very thick, very sticky (glue like in nature) and are difficult to spread onto the skin.

The compositions of the present invention for forming a visible and distinctive cosmetic film on the skin of a subject may optionally contain active ingredients, humectants/plasticizers, other polymers and thickeners, dyes/pigments, surfactants/emulsifiers, hydrocarbons or other oils, fragrances, chelating agents, UV absorbers, antioxidants, preservatives, and mixtures thereof.

Pullulan is a polysaccharide produced from a cultivated fungus of Aureobasidium pullulans (pulluaria pullulans). Pullulan is an alpha-glucan mainly constituted of maltrotriose as repeating units linearly joined through alpha-1,6-glycosidic linkages, not branched. Pullulan is also amorphous, edible and naturally degradable. It is readily soluble in cold or warm water and forms clear, viscous solutions therewith. Its industrial production has been developed in Japan by Hayashibara Biochemical Laboratories, Inc., from the fermentation of partially hydrolyzed starch. Pullulan may also be produced using sucrose, sugar cane, milk, potato and other sources for the needed carbohydrates. Preferably, the pullulan used in the compositions of the invention will exhibit a molecular weight of 200,000 to 2,000,000 Daltons.

Solvents suitable for use in the compositions of the present invention include, but are by no means limited to, water, alcohol, glycol and mixtures thereof. Preferably, the compositions of the present invention include water as the solvent.

Active ingredients suitable for use in the compositions of the present invention include, but are by no means limited to, oil- or water-soluble synthetic or natural active ingredients conventionally used in cosmetics. Preferably, the active ingredients may include alpha- and beta-hydroxy acids and their derivatives; botanical extracts, botanical oils and their derivatives; vitamins, for example, vitamin C, vitamin B, vitamin E, vitamin A, their salts, esters, alcohols and acid forms thereof; marine products and their derivatives; and topical over the counter active ingredients. The compositions of the present invention may include as a preferred active agent a mixture of bearberry extract, zinc sulfate, sodium salicylate and ethoxydiglycol effective for combating acne. An active mixture of these constituents suitable for use in the compositions of the present invention is available from Gattefosse under the trademark Gatuline Equalizing. Other preferred active agents for use in the compositions of the present invention include keratolytic agents effective for combating acne. Preferably, the compositions of the present invention may contain 0.01 to 10 wt % active ingredients, more preferably 0.10 to 8.0 wt %.

Humectants/plasticizers suitable for use in the compositions of the present invention include, but are by no means limited to, any water-binding ingredients generally used in cosmetics. Preferably, the humectants/plasticizers may include glycols, for example glycerin, propylene glycol and 1,3-butylene glycol; sorbitol, sodium polycarboxcylic acid, and sodium citrate. Most preferably, the humectant/plasticizer may include polypropylene glycol-10 methyl glucose ether. A polypropylene glycol-10 methyl glucose ether suitable for use in the compositions of the present invention is available from Amercol under the trademark Glucam P-10. Preferably, the compositions of the present invention may contain 0.5 to 20 wt % humectants/plasticizers. Humectants/plasticizers may be added to the compositions of the present invention to modify the properties and wear time of the resultant film produced on the skin of the subject when using the compositions.

Other polymers and thickeners suitable for use in the compositions of the present invention include, but are by no means limited to, any polymer or thickener conventionally used in cosmetics. Preferably, the thickeners may include xanthan gum; cellulose; polyvinyl pyrolidone; carbomer; mixtures such as (1) ammonium polyacrylate, isohexadecane and polyethylene glycol-40 castor oil (an example of such a mixture suitable for use with the present invention is available from Seppic under the trademark Simulgel A); (2) polyacrylamide, polydecene and ethoxylated lauryl alcohol (an example of such a mixture suitable for use with the present invention is available from C.I.T. Sear under the trademark Ceragel EZ-7); (3) polyacrylamide, C₁₃₋₁₄ isoparaffin and ethoxylated lauryl alcohol (an example of such a mixture suitable for use with the present invention is available from Seppic under the trademark Sepigel 305); (4) polyquaternium 32 and mineral oil (an example of such a mixture suitable for use with the present invention is available from Ciba under the trademark Salcare SC-92); and (5) acrylates and C₁₀₋₃₀ acyl acrylate crosspolymer (an example of such a mixture suitable for use with the present invention is available from Noveon under the trademark Carbopol 1342). Preferably, the compositions of the present invention may contain 0.1 to 10 wt % thickeners. Thickeners may be added to the compositions of the present invention to modify the viscosity of the composition, the feel of the composition and the film formed therewith when applied to the skin of a subject, the spreadability of the composition and the strength of the film formed by the composition upon application to the skin of a subject.

Dyes/pigments suitable for use in the compositions of the present invention include, but are by no means limited to, any inorganic dyes, organic dyes, pigments and opacifiers conventionally used in cosmetics. Preferably, the dyes/pigments may include titanium dioxide, zinc oxide, iron oxide, D&C and FD&C dyes. The compositions of the present invention may contain an opacifier. An opacifier suitable for use in the compositions of the present invention may preferably contain a mixture of styrene and polyvinyl pyrolidone copolymer (an example of such a mixture suitable for use with the present invention is available from ISP under the trademark Polectron 430). Preferably, the compositions of the present invention may contain 0.01 to 10 wt % dyes/pigments.

Surfactants/emulsifiers suitable for use in the compositions of the present invention include, but are by no means limited to, any anionic, cationic and nonionic surfactants and emulsifiers conventionally used in cosmetics. Preferably, the surfactants/emulsifiers may include ethoxylated alcohols, sodium lauryl sulfate and polyquaternium-31. Preferably, the compositions of the present invention may contain 0.5 to 10 wt % surfactants/emulsifiers. Surfactants/emulsifiers may be added to the compositions of the present invention to stabilize the composition and/or to improve the spreadability of the composition onto the skin of the subject.

Hydrocarbons or other oils suitable for use in the compositions of the present invention include, but are by no means limited to, iso-paraffin, mineral oil, isododecane, almond oil and other natural oils. Preferably, the compositions of the present invention may contain 0.5 to 10 wt % hydrocarbons or other oils.

Fragrances suitable for use in the compositions of the present invention include, but are by no means limited to, lime, peppermint and citrus. Preferably, the compositions of the present invention may contain 0.1 to 5 wt % fragrances. Fragrances may be added to the compositions of the present invention to support a marketing concept and/or to mask any unpleasant odors associated with the composition or natural to the cite of application on the subject's skin.

Chelating agents suitable for use in the compositions of the present invention include any chelating agents conventionally used in cosmetics. Preferably, the chelating agents may include ethylenediaminetetraacetic acid, ethylenediaminetetraacetic acid-disodium salt and ethylenediaminetetraacetic acid-tetrasodium salt. Preferably, the compositions of the present invention may contain 0.01 to 1 wt % chelating agents. Chelating agents may be added to the compositions of the present invention to bind metal ions and to promote the overall stability of the compositions.

UV absorbers suitable for use in the compositions of the present invention include any water and/or oil soluble sunscreen conventionally used in cosmetics. Preferably, the UV absorbers may include benzophenone-3 and benzophenone-4. Preferably, the compositions of the present invention may contain 0.1 to 4 wt % UV absorbers. UV absorbers may be added to the compositions of the present invention to protect the color of the composition from the deleterious effects of exposure to ultra-violet light.

Antioxidants suitable for use in the compositions of the present invention include any antioxidants conventionally used in cosmetics. Preferably, the antioxidants may include tocopherol, tocopherol acetate, propyl gallate, butylated hydroxyanisole and butylated hydroxytoluene. Preferably, the compositions of the present invention may contain 0.05 to 3 wt % antioxidants. Antioxidants may be added to the compositions of the present invention to combat the deleterious effects of oxidation.

Preservatives suitable for use in the compositions of the present invention include any preservative or preservative system conventionally used in cosmetics. Preferably, preservatives suitable for use in the compositions of the present invention may include a mixture of phenoxyethanol, methylparaben, isopropylparaben, isobutyparaben and butylparaben (an example of such a mixture suitable for use with the present invention is available from ISP/Sutton under the trademark LiquaPar Optima). Preferably, the compositions of the present invention may contain 0.01 to 3 wt % preservatives. Preservatives may be added to the compositions of the present invention to combat microbiological contamination.

The compositions of the present invention for forming a visible and distinctive cosmetic film on the skin of a subject preferably contain: 3 to 15 wt % pullulan, 20 to 97 wt % solvent, 0.01 to 10 wt % active agent, 0.5-20 wt % humectant, 0.1 to 10 wt % other polymers and thickener, 0.01 to 10 wt % pigment, 0.5 to 10 wt % emulsifier, 0.5 to 10 wt % hydrocarbon, 0.1 to 5 wt % fragrance, 0.01 to 1 wt % chelating agent, 0.1 to 4 wt % UV absorber, 0.05 to 3 wt % antioxidant and 0.01 to 3 wt % preservative. Preferably, the composition may be provided as a semi-liquid gel or as a roll-on.

The method of the present invention for forming a visible and distinctive cosmetic film on the skin of a subject preferably includes: applying to the skin of the subject a composition containing: 3 to 15 wt % pullulan, more preferably 3 to 10 wt % pullulan, most preferably 10 wt % pullulan. Preferably, the method may include: applying to the skin of the subject a composition containing: 3 to 15 wt % pullulan, 20 to 97 wt % solvent, 0.01 to 10 wt % active agent, 0.5-20 wt % humectant, 0.1 to 10 wt % other polymers and thickener, 0.01 to 10 wt % pigment, 0.5 to 10 wt % emulsifier, 0.5 to 10 wt % hydrocarbon, 0.1 to 5 wt % fragrance, 0.01 to 1 wt % chelating agent, 0.1 to 3 wt % UV absorber, 0.05 to 3 wt % antioxidant and 0.01 to 3 wt % preservative. Under the method of the present invention, the composition may dry to a visible and distinctive film on the skin of the subject in 15 minutes or less following application to the skin, more preferably in 3 to 15 minutes.

### EXAMPLES

The preferred embodiments of the present invention will now be further described through the following example set forth hereinbelow which is intended to be illustrative of the preferred embodiments of the present invention and is not intended to limit the scope of the invention as set forth in the appended claims.

### Example 1

A composition of the present invention for forming a visible and distinctive cosmetic film on the skin of a subject with the formula presented in Table 1 was prepared.

TABLE 1
Gel-Patch with anti-irritant

| Ingredient | % w/w |
|---|---|
| Water | QS |
| Na₂EDTA | 0.05 |
| Mixture: acrylates and C₁₀₋₃₀ acyl acrylate | 0.60 |
| crosspolymer (Carbopol 1342) | |
| Pullulan | 10.00 |
| Mixture: ammonium polyacrylate, | 1.00 |
| isohexadecane and PEG-40 castor oil | |
| (Simugel A) | |
| DL-alphatochopherol | 0.10 |
| Mixture: phenoxyethanol, methylparaben, | 0.70 |
| isopropylparaben, isobutylparaben and | |
| butylparaben (LiquaPar Optima) | |
| bisabolol | 1.00 |
| NaOH (10%) | 1.50 |

The composition identified in Table 1 was prepared as follows:

(a) dispersing the Na₂EDTA and the mixture of acrylates and C₁₀₋₃₀ acyl acrylate crosspolymer (Carbopol 1342) in water;

(b) mixing the product of (a) for about 20 minutes;

(c) adding the pullulan into the mixture of (b);

(d) mixing the product of (c) for about 25 minutes until uniform;

(e) adding the mixture of ammonium polyacrylate, isohexadecane and PEG-30 castor oil (Simugel A) to the product of (d) and mixing until uniform;

(f) adding the DL-alphatochopherol to the product of (e) and mixing until uniform;

(g) adding the mixture of phenoxyethanol, methylparaben, isopropylparaben, isobutylparaben and butylparaben (LiquaPar Optima) to the product of (f) and mixing until uniform;

(h) adding the bisabolol to the product of (g) and mixing until uniform; and

(i) adding the NaOH to the product of (h) and mixing until uniform.

The product composition was a semi-liquid. The product composition was found to form a comfortable and elegant, visible and distinctive film when applied to the skin and allowed to dry.

### Example 2

A composition of the present invention for forming a visible and distinctive cosmetic film on the skin of a subject with the formula presented in Table 2 was prepared.

TABLE 2
Gel-Patch with salycilic acid

| Ingredient | % w/w |
|---|---|
| Water | QS |
| Na₂EDTA | 0.05 |
| Mixture: acrylates and C₁₀₋₃₀ acyl acrylate | 0.60 |
| crosspolymer (Carbopol 1342) | |
| Pullulan | 10.00 |
| Mixture: ammonium polyacrylate, | 1.00 |
| isohexadecane and PEG-40 castor oil | |
| (Simugel A) | |
| DL-alphatochopherol | 0.10 |
| Mixture: phenoxyethanol, methylparaben, | 0.70 |
| isopropylparaben, isobutylparaben and | |
| butylparaben (LiquaPar Optima) | |
| bisabolol | 1.00 |
| Salicylic Acid | 0.50 |
| Ethyl Alcohol | 2.00 |

The composition identified in Table 1 was prepared as follows:

(a) dispersing the Na₂EDTA and the mixture of acrylates and C₁₀₋₃₀ acyl acrylate crosspolymer (Carbopol 1342) in water;

(b) mixing the product of (a) for about 20 minutes;

(c) adding the pullulan into the mixture of (b);

(d) mixing the product of (c) for about 25 minutes until uniform;

(e) adding the mixture of ammonium polyacrylate, isohexadecane and PEG-30 castor oil (Simugel A) to the product of (d) and mixing until uniform;

(f) adding the DL-alphatochopherol to the product of (e) and mixing until uniform;

(g) adding the mixture of phenoxyethanol, methylparaben, isopropylparaben, isobutylparaben and butylparaben (LiquaPar Optima) to the product of (f) and mixing until uniform;

(h) adding the bisabolol to the product of (g) and mixing until uniform; and,

(i) adding a uniform mixture of the ethanol and salicylic acid to the product of (h) and mixing until uniform.

The product composition was a semi-liquid. The product composition was found to form a comfortable and elegant, visible and distinctive film when applied to the skin and allowed to dry.

### Example 3

### Skin Permeation Experiment

A description of the materials and test parameters used to perform a skin permeation test used to determine the salicylic acid permeation from a gel-patch with salicylic acid of Example 2 are provided in Table 3.

TABLE 3

| A. Materials or Test Parameter | II. Description |
|---|---|
| Human Allograft Skin-Source | Female Human Cadaver Skin with an |
| Intermount Tissue Center, | approximate thickness of |
| Salt Lake City, Utah | 0.010-0.015 inches |
| Receptor Fluid | Phosphate Buffered Normal Saline with |
| | 20% PEG 400 |
| Permeation Apparatus | In-line Auto Sampling System |
| Sampling times | 15, 30, 60, 120, 180, 240 minutes |
| Salicylic Acid Analysis | HPLC method |

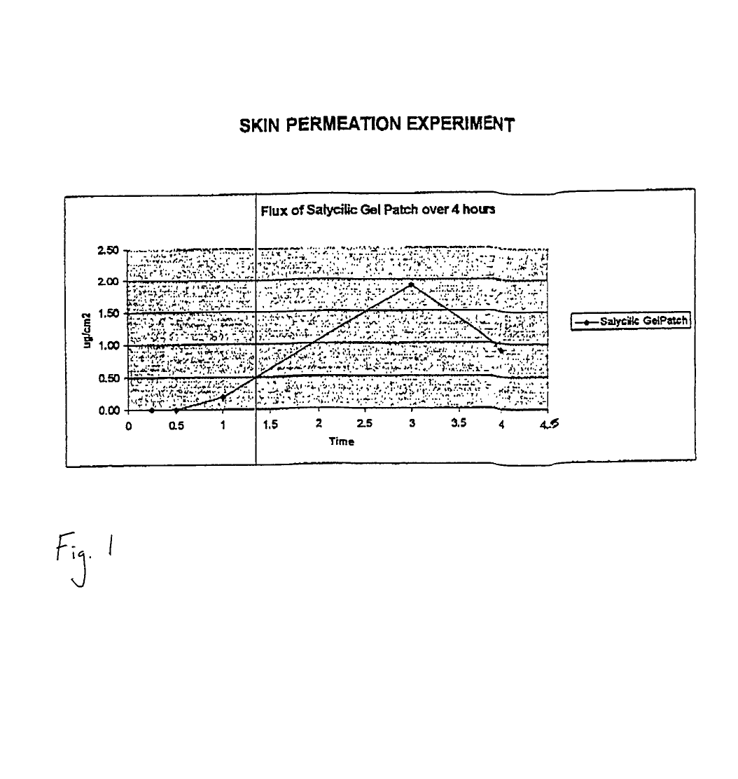

A 2-mil thickness dried film of Example 2 was used for this test. The film was applied to the epidermal side of the cadaver skin, which was then placed into an in-line auto sampling apparatus. Receptor fluid was continuously passed through the cells of the auto sampling device bathing the dermal side of the cadaver akin and received the “permeated drud” as it passed. The collection sampe was then analyzed using an HPLC for drug content. The results of this test are presented in FIG. 1.

The present invention having been disclosed in connection with the foregoing preferred embodiments and examples, additional embodiments will now be apparent to persons skilled in the art. The present invention is not intended to be limited to the preferred embodiments specifically mentioned, and accordingly reference should be made to the appended claims rather than the foregoing discussion, to assess the spirit and scope of the present invention in which exclusive rights are claimed.

We claim:

1. A composition for forming a cosmetic film, comprising 3 to 15 wt % pullulan.

2. The composition of claim 1, wherein the composition comprises 3 to 10 wt % pullulan.

3. The composition of claim 1, wherein the composition comprises 10 wt % pullulan.

4. The composition of claim 1, wherein the composition is a semi-liquid gel or a roll-on.

5. The composition of claim 1, wherein the pullulan has a molecular weight of 200,000 to 2,000,000 Daltons.

6. The composition of claim 1, wherein the composition further comprises 20 to 97 wt % solvent.

7. The composition of claim 6, wherein the solvent is selected from the group consisting of water, alcohol, glycol and mixtures thereof.

8. The composition of claim 1, wherein the composition further comprises 0.01 to 10 wt % active agent.

9. A composition for forming a cosmetic film, comprising: 3 to 15 wt % pullulan, 20 to 97 wt % solvent, 0.01 to 10 wt % active agent, 0.5-20 wt % humectant, 0.1 to 10 wt % other polymers and thickener, 0.01 to 10 wt % pigment, 0.5 to 10 wt % emulsifier, 0.5 to 10 wt % hydrocarbon, 0.1 to 5 wt % fragrance, 0.01 to 1 wt % chelating agent, 0.1 to 3 wt % UV absorber, 0.05 to 3 wt % antioxidant and 0.01 to 3 wt % preservative.

10. The composition of claim 9, wherein the solvent is selected from the group consisting of water, alcohol, glycol and mixtures thereof.

11. The composition of claim 9, wherein the active agent is selected from the group consisting of oil soluble or water soluble natural or synthetic ingredients.

12. The composition of claim 9, wherein the active agent is selected from the group consisting of alpha- and beta-hydroxy acids and derivatives thereof; vitamins and the salts, esters, alcohols and acid forms thereof; marine products and their derivatives; and over the counter topical ingredients.

13. The composition of claim 9, wherein the humectant is selected from the group consisting of glycols, sorbitol, sodium polycarboxcylic acid, sodium citrate and mixtures thereof.

14. The composition of claim 9, wherein the thickener is selected from the group consisting of xanthan gum, cellulose, polyvinyl pyrolidone, carbomer and mixtures thereof.

15. The composition of claim 9, wherein the thickener is selected from the group mixtures consisting of:
(a) a mixture of ammonium polyacrylate, isohexadecane, polyethylene glycol and castor oil;
(b) a mixture of polyacrylamide, polydecene, and ethoxylated lauryl alcohol;
(c) a mixture of polyacrylamide, C₁₃₋₁₄ isoparaffin, and ethoxylated lauryl alcohol;
(d) a mixture of polyquaternium-32, and mineral oil; and,
(e) a mixture of acrylates and C₁₀₋₃₀ acyl acrylate crosspolymer.

16. The composition of claim 9, wherein the pigment is selected from the group consisting of: titanium dioxide, zinc oxide, iron oxide, Drug and Cosmetic (D&C) dyes and Food, Drug and Cosmetic (FD&C).

17. The composition of claim 9, wherein the emulsifier is selected from the group consisting of: ethoxylated alcohols, sodium lauryl sulfate, and polyquaternium-31.

18. The composition of claim 9, wherein the hydrocarbons is selected from the group consisting of: iso-paraffin, mineral oil, isododecane, almond oil and other natural oils.

19. The composition of claim 9, wherein the chelating agent is selected from the group consisting of: ethylenediaminetetraacetic acid, ethylenediaminetetraacetic acid-disodium salt and ethylenediaminetetraacetic acid-tetrasodium salt.

20. The composition of claim 9, wherein the UV absorber is selected from the group consisting of: benzophenone-3 and benzophenone-4.

21. The composition of claim 9, wherein the antioxidant is selected from the group consisting of tocopherol, tocopherol acetate, propyl gallate, butylated hydroxyanisole, and butylated hydroxytoluene.

22. The composition of claim 9, wherein the preservative is selected from the group consisting of: methylparaben, propylparaben and phenoxyethanol.

23. A method for forming a cosmetic film on the skin of a subject, comprising: applying to the skin of the subject a composition comprising: 3 to 15 wt % pullulan.

24. A method for forming a cosmetic film on the skin of a subject, comprising: applying to the skin of the subject a composition comprising: 3 to 15 wt % pullulan, 20 to 97 wt % solvent, 0.01 to 10 wt % active agent, 0.5-20 wt % humectant, 0.1 to 10 wt % other polymers and thickener, 0.01 to 10 wt % pigment, 0.5 to 10 wt % emulsifier, 0.5 to 10 wt % hydrocarbon, 0.1 to 5 wt % fragrance, 0.01 to 1 wt % chelating agent, 0.1 to 3 wt % UV absorber, 0.05 to 3 wt % antioxidant and 0.01 to 3 wt % preservative.
